(19) United States
(12) Patent Application Publication
(10) Pub. No.: US 2002/0037899 A1
(43) Pub. Date: Mar. 28, 2002

(54) Compositions containing an inhibitor of dihydrofolate reductase and a folate

(76) Inventors: Joseph E. Baggott, Mountain Brook, AL (US); Sarah L. Morgan, Vestavia, AL (US)

Correspondence Address: Glenna Hendricks, Esq., P.O. Box 2509, Fairfax, VA, 22031-2509, US

(21) Appl. No.: 09/924,949

(22) Filed: Aug. 9, 2001

Related U.S. Application Data
(60) Provisional application No. 60/224,025, filed on Aug. 10, 2000.

(51) Int. Cl. A61K 31/525; A61K 9/48; A61K 9/20; A61K 31/519

(52) U.S. Cl. 514/251; 424/451; 424/464; 514/258

(57) ABSTRACT

The deleterious symptoms of autoimmune disease in a patient suffering from symptoms of autoimmune disease can be relieved by by co-administration of a cell-mediated immune inhibiting effective amount of at least one inhibitor of dihydrofolate reductase and a folate wherein the folate is formulated for delayed release in a carrier.

This application takes priority from Provisional Patent Application No. 60/224,025, filed Aug. 10, 2000.

### FIELD OF THE INVENTION

This invention relates to the field of treatment of autoimmune related disease conditions using compositions containing cell-mediated immune inhibiting doses of inhibitors of dihydrofolate reductase and folic acid and their analogues.

### BACKGROUND OF THE INVENTION

Methotrexate is an antimetabolite compound of the formula:

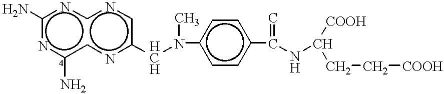

and has been used for treatment of various malignancies since the 1960's. It is a folate antagonist. Methotrexate has also been used in lower dosage in treatment of autoimmune diseases such as psoriasis and rheumatoid arthritis. Because methotrexate interferes with folic acid metabolism when given in high dosage as an antineoplastic agent, folinic acid, a metabolite of folic acid, is often given as a “rescue” agent to rescue normal cells from the toxic effects of methotrexate. However, the much lower doses used as suppressors of autoimmune diseases are such that, with administration of folic acid, most of the toxic effects can be avoided. Under these circumstances, the methotrexate can be given in conjunction with folic acid and its analogues.

Until the present, it has been necessary to give the folio acid and methotrexate in separate compositions. The folic acid and the methotrexate are given on different days. For example, the folic acid may be administered daily 3-5 times a week and the methotrexate administered on a day when the folic acid is not given. This method of dosing presents problems, since it is essential that the patient remember dosage and schedule when each active agent is to be taken. It is not advisable to have the patient absorb both the folic acid (a vitamin) and the methotrexate (an antivitamin) simultaneously.

Several means of controlling the time and anatomical location of drug release are known, including chemical means such as substitutions which act as protective groups. For example, the folic acid can be substituted with protective groups that are stable at low pH, but will be removed at higher pH in the small intestine. It is also possible to encapsulate the folic acid in, for example, liposomes or granules.

Barry, et al, in U.S. Pat. No. 5,051,263, which is incorporated herein by reference in its entirety, teaches preparation of granules which may be coated. The coated granules range in size from 0.5 to 2.5 mm.

Sherman, U.S. Pat. No. 5,879,714, which is incorporated by reference herein in its entirety, teaches preparation of pharmaceutical compositions wherein the active agents are formulated with a water-insoluble polymer in a molten carrier to provide granules having controlled delivery.

### SUMMARY OF THE INVENTION

It is the purpose of this invention to provide medicinal compositions containing inhibitors of dihydrofolate reductase in dosage appropriate for use in treating diseases related to cell-mediated immune responses along with folic acid or its analogues in a single dosage form. In such compositions the folic acid is prepared by means known in the art such as incorporation in granules or liposomes. In short, the method of the invention comprises relieving deleterious symptoms of autoimmune disease in a patient suffering from symptoms of autoimmune disease by administration of a composition comprising a cell-mediated immune inhibiting effective amount of at least one inhibitor of dihydrofolate reductase and a folate wherein the folate in said formulation is formulated for delayed release in a carrier. In a second embodiment of the invention, a single dosage package containing (1) a composition containing a cell-mediated immune inhibiting effective amount of at least one inhibitor of dihydrofolate reductase and (2) a composition containing a folate wherein the folate is in a delayed release carrier system.

### DETAILED DESCRIPTION OF THE INVENTION

Methotrexate, an antivitamin of folic acid, is frequently given for treatment of autoimmune-related diseases such as rheumatoid arthritis and psoriasis. Other diseases with autoimmune etiologies in which the antifolate, methotrexate, have been used include psoriatic arthritis, asthma, bullous pemphigoid, cerebral vasculitis, cochlear vestibular disorders, dermatomyositis, Felty's syndrome, graft-versus host disease, idiopathic granulomatous hepatitis, inflammatory bowel disease (Crohn's and ulcerative colitis), multiple sclerosis, mycosis fungoides, pemphigus, vulgaris, pityriasis rubra, polymyalgia rheumatica, polymyositis, primary biliary cirrhosis, primary sclerosing cholangitis, psoriatic arthritis, pyoderma gangrenosum, Reiter's syndrome, rheumatoid arthritis, sarcoidosis, sclerosing cholangitis, Sezary syndrome, Takayasu's disease, uveitis, vasculitis, and Wegener's granulomatosis. There are other diseases classified as autoimmune diseases such as, for example, Sjogren's Syndrome and lupus erythematosis for which the compositions and methods of the invention would be appropriate. Folic acid is often given to patients who have been previously dosed with the antifolate, methotrexate. While it has been demonstrated that folic acid can lessen the toxic effects of methotrexate, the folic acid must not be absorbed into the blood stream at the same time as the methotrexate, since their effects are counteractive. A recent analysis evaluating the efficacy of folic acid and folinic acid in reducing toxic effects of methotrexate on the gastrointestinal tract showed that there was a 79% reduction in mucosal and gastrointestinal side effect when the folic acid or folinic acid was administered to rheumatoid arthritis patients receiving methotrexate. However, when high dosages of folinic acid were administered there was an increase in disease symptoms such as painful joints.

It would be beneficial if folic acid and/or one or more of its analogues, including salts thereof, could be administered in the same formulation as the inhibitor of dihydrofolate reductase. However, it is not desirable to have the two medications absorbed into the systemic circulation at the same time. Hence, this invention provides formulations containing inhibitors of dihydrofolate reductase (antifolates) and folic acid, its salts or analogues, including protected forms of folic acid, wherein the folate is in a form that will be absorbed subsequent to the absorption of the antifolate. Inhibitors of dihydrofolate reductase, in addition to methotrexate, include 10-deazaminopterin, aminopterin and trimethoprim. However, the most widely used antifolate at the present time is methotrexate. Hence, Methotrexate is the compound exemplified in this disclosure.

Dihydrofolate reductase is a critical enzyme of folate metabolism, which reduces folate to its active coenzyme form. A folate is a vitamin that is involved in maintenance of normal cellular metabolism and cell division. The term “folate” is often used as a generic term for the family of folate coenzymes (folic acid, folinic acid, 5-methyl tetrahydrofolate, etc). Folate coenzymes are essential for biosynthesis of both DNA and RNA. The folate most frequently administered is folic acid. (Folinic acid is discussed above.) The availability of one dosing form containing both folic acid and methotrexate formulated in such a manner that the active agents would not be released into the blood stream simultaneously, since the activity of one counteracts the activity of the other, would obviate the need for separate dosing.

The folic acid and esters thereof may be formulated in carrier systems known in the art such as granules, microcapsules or liposomes. Because of cost considerations, granules or microcapsules would be more likely choices. However, the most likely form for use in such compositions would involve granules which are formulated for timed-release or delayed-release system. (As used herein, “delayed-release” relates to any composition whose release is retarded, including sustained release, which causes the retardation in time of release of the active agent so that it can effect the expected result in the patient.) Folic acid has a limited solubility in water (1.6 mg per ml) and is stable to heating at a temperatures below 200° C. Hence, granules containing folates (particularly, folic acid) formed in accord with the teachings of the Barry or Sherman patent could be prepared for inclusion in capsules or tablets which also would contain at least one antifolate such as methotrexate. Other ingredients such as, for example, cellulose, starch, sodium starch glycolate or croscarmellose sodium, which will absorb water and swell so as to cause disintegration of the tablet, might also be present. Alternatively, or in addition, the other ingredients may include a water—soluble material, such as, for example, lactose, mannitol, sorbitol, methylcellulose, or hydroxypropyl-methylcellulose (which is available in a variety of grades having various degrees of hydroxypropyl substitution and various mean molecular weights), which will dissolve in gastrointestinal fluid thereby again causing the tablet to disintegrate and to release the granules and other active agents.

For pediatric use, the compositions containing the active agents, including the active agents in delayed-release form, may be provided, for example, as easily crushed tablets for addition to food or as suspensions (which may be in the form of soft gels) that can be swallowed more easily.

Dosage of the pharmaceutical preparations would be in accord with that suggested in prior literature. For example, for adult use, dosage of methotrexate being present at about 2 to 10 mg and folic acid at about 10 to 75 mg would be a preferred range. The dosage and ratio of the agents in compositions containing both the antifolate and folate would vary depending on differential absorption resulting from the methods of formulation of the particular pharmaceutical preparation.

In another aspect of the invention, a composition containing the antifolate in easily absorbed form could be co-packaged with a folate which is formulated as a delayed release product. The two forms would be packaged together in appropriate dosages to be taken concurrently. This would avoid confusion relating to the which medication is to be taken any particular day. For example, packaging containing the appropriate dosage of both methotrexate for treatment of the autoimmune-related disease and folic acid in delayed release form could be packaged together for concurrent administration. This is particularly important, since prior dosage errors wherein larger doses of antifolate appropriate for treatment of malignancies have been administered without appropriate dosage of folate to counteract the deleterious side effect of the antifolate. Such errors could be avoided by preparation of packaging containing, for co-administration, appropriate dosage of the antifolate with the appropriate accompanying dose of folate wherein the folate is in a delayed release formulation.

### EXAMPLE 1

The following composition is made in accord with the teachings of Barry, U.S. Pat. No. 5,051,263, which is incorporated herein by reference. A composition is prepared by dry mixing

5% powdered folic acid

10% carbopol 934P

85% Avicel PH101.

After mixing, the product is added to water until a cohesive product is formed. The product is extruded to produce slugs of about 1 mm diameter and 3 mm length. The slugs are then passed through a spheronizer to create granules, which are then dried to a consistent weight. The final product will contain 5% folic acid.

The Example refers to the use of CARBOPOL™ 934 P which is a commercially available brand of carbomer. In addition to the pharmacologically active substances and carbomer, the formulations also preferably contain bulking agents such as microcrystalline cellulose. This is a well known form of cellulose which is partially depolymerized. A particularly suitable microcrystalline cellulose is sold under the name AVICEL™ (a registered trade mark). However, other conventional bulking agents may also be used, as will be readily apparent to those skilled in the art.

### EXAMPLE 2

The following composition is prepared in accord with the teachings of U.S. Pat. No. 5,879,714. The following ingredients were used:

| | |
|---|---|
| Polyethylene glycol 8000 | 97 gm |
| Powdered folic acid | 33 gm |
| Steric acid | 5 gm |
| Eudragit RSPO | 25 gm |

The polyethylene glycol 8000 is melted and further heated to about 120° C. The folic acid is added with stirring for purposes of dispersing the folic acid throughout the mixture. The steric acid is then added and stirred until it is melted, after which the EUDRAGIT™ RSPO is added and stirred until fully dispersed. The molten mixture is then allowed to cool and solidify, after which it is ground into granules. The granules contain 20.6% folic acid in a delayed-release preparation. (EUDRAGIT™ is made by Rohm Pharma GMBH.)

Capsules are prepared containing 7.5 mg methotrexate, granules containing 50 mg folic acid with sufficient filler to provide a total amount of 0.5 gm.

Other active agents such as antibiotics, steroids and antihistamines may be added to compositions. Furthermore, additives used in the pharmaceutical arts such as flavorants, preservatives and coloring agents may be used in preparation of the compositions of the invention. The use of distinctive coloration for different dosages would be particularly helpful in these preparations, since dosage of antifolates varies greatly, depending on whether the medication is administered for treatment of malignancy or of autoimmune disease.

It should be remembered that the folic acid incorporated into the biologically active pool of tetrahyrofolates will always be less than the amount administered because of the partial inhibition of dihydrofolate reductase by the antifolate. Hence, the dosage of folic acid administered will be relatively high. The ratio of methotrexate:folic acid (w/w) will vary greatly as within the range of 1:0.01 to 1:50, with the more preferred range being 1:1-1:25. Other antifolate:folate ranges will be approximately the same, depending on the condition and age of the patient.

The dosage of methotrexate in adults for treatment of autoimmune-related pathologies would be about 1 to 30 mg per week, while the dose of folic acid would be from 1 to about 500 mg per week. A preferred dosage would be 2.5-25 mg per week of methotrexate and about 3-100 mg per week of folic acid, with the more preferred dosage of folic acid being 10 mg to 100 mg per week with the dosage divided so that the compositions would be administered at least one day of the week. Hence, preferred compositions containing methotrexate and folic acid as the active agents would contain 0.75 to 12.5 mg methotrexate and 5 to 50 mg. folic acid.

What we claim is:

1. A composition of matter comprising an antifolate and a folate wherein the folate is in a delayed release carrier system in a carrier.

2. The composition of claim 1 wherein the folate is folic acid and the antifolate is methotrexate.

3. The composition of claim 1 which is a tablet.

4. The composition of claim 1 which is a capsule.

5. The composition of claim 2 containing folic acid in a delayed-release granular form.

6. The composition of claim 1 comprising, as active agents, methotrexate and folic acid in a methotrexate:folic acid ratio of 1:0.01 to 1:50 wherein the folate is in the form of a delayed release preparation.

7. The composition of claim 6 wherein the ratio of methotrexate:folic acid is in the range of 1:1-1:25.

8. The composition of claim 1 wherein the antifolate selected from among methotrexate, 10-deazaminopterin, aminopterin and trimethoprim.

9. The composition of claim 2 containing 5 to 50 mg folic acid and 0.75 to 12.5 mg methotrexate.

10. A method of relieving deleterious symptoms of autoimmune disease in a patient suffering from symptoms of autoimmune disease by administration of a composition comprising a cell-mediated immune inhibiting effective amount of at least one inhibitor of dihydrofolate reductase and a folate wherein the folate in said formulation is formulated for delayed release in a carrier.

11. The method of claim 10 wherein the inhibitor of dihydrofolate reductase is methotrexate and the folate is folic acid or a salt thereof.

12. The method of claim 11 wherein the composition administered contains 0.75-12.5 mg methotrexate and 5-50 mg folic acid or a salt thereof.

13. The method of claim 10 wherein said composition is administered 1 to 2 days a week.

14. The method of claim 12 wherein the composition contains, as the folate, folic acid.

15. A single dosage package containing (1) a composition containing a cell-mediated immune inhibiting effective amount of at least one inhibitor of dihydrofolate reductase and (2) a composition containing a folate wherein the folate is in a delayed release carrier system.

16. The single dosage package of claim 15 wherein the inhibitor of dihydrofolate reductase is methotrexate and the folate is folic acid.

17. The dosage package of claim 15 wherein the methotrexate is present at a dosage of 0.75-12.5 mg methotrexate and a composition containing 5-50 mg folic acid.
